(19) United States
(12) Patent Application Publication
(10) Pub. No.: US 2011/0245600 A1
(43) Pub. Date: Oct. 6, 2011

(54) SOLID-STATE IMAGE PICKUP DEVICE AND ENDOSCOPIC DEVICE

(75) Inventors: Shuichi ISHII, Ashigarakami-gun (JP); Takashi YASHIRO, Ashigarakami-gun (JP); Kazuaki TAKAHASHI, Ashigarakami-gun (JP)

(73) Assignee: FUJIFILM Corporation, Tokyo (JP)

(21) Appl. No.: 13/017,621

(22) Filed: Jan. 31, 2011

(30) Foreign Application Priority Data
Mar. 31, 2010 (JP) 2010-082254

(51) Int. Cl. A61B 1/04; A61B 1/018; H04N 7/18

(52) U.S. Cl. 600/104; 600/112; 348/76; 348/E07.085

(57) ABSTRACT

A solid-state image pickup device, comprising:
a solid-state image pickup element;
a circuit-board main body to which the solid-state image pickup element is connected;
a shield piece consecutively connected to the circuit-board main body, capable of being folded, and having a shield pattern disposed; and
a signal cable which is connected to a connection terminal disposed on the circuit-board main body and transmits an input/output signal to the solid-state image pickup element, wherein
the shield piece is folded at least to one face side of a region where the connection terminal is formed.

### BACKGROUND OF THE INVENTION

1. Field of the Invention

The present invention relates to a solid-state image pickup device incorporated in a distal end of an endoscope insertion portion and an endoscopic device.

2. Description of the Related Art

In the medical field, diagnoses using an endoscopic device (electronic endoscope) are widely performed. In the endoscopic device, a solid-state image pickup element (such as a CCD image pickup element) is incorporated in a distal end of an insertion portion to be inserted into a subject body, and a signal cable connected to a circuit board on which the solid-state image pickup element is mounted is connected to a connector connected to a processor (signal processor) through the insertion portion. By applying signal processing by the processor to an image pickup signal outputted from the solid-state image pickup element, an image (endoscopic image) of an observed portion can be observed by a monitor device. Also, a forceps channel through which a treatment instrument is inserted is disposed in the insertion portion of the endoscopic device, and the device might be used by inserting en electric treatment instrument such as a high-frequency knife through this forceps channel.

Size reduction and high-density mounting have been promoted for the insertion portion of the endoscopic device, and measures against noise in the insertion portion have been one of important technical problems.

For example, Japanese Patent Application Laid-Open No. 2008-237842 discloses a technology in which a shield member is applied to a portion in a transmission path for solid-state image pickup element that overlaps a transmission path for ultrasonic signal in order to prevent an influence of the noise from the transmission path for solid-state image pickup element to the transmission path for ultrasonic signal.

Also, Japanese Patent Application Laid-Open No. 2010-35755 discloses a technology in which a resin tube constituting the forceps channel is covered by a metal plating layer in order to protect from high-frequency noise emitted from the high-frequency knife inserted through the forceps channel.

However, with the prior-art technologies disclosed in Japanese Patent Application Laid-Open No. 2008-237842 and Japanese Patent Application Laid-Open No. 2010-35755, a conductor is in a bare state at the distal end of the signal cable connected to a connection terminal of a circuit board, and a measure against noise for this portion is not examined at all. Thus, there is a problem that the noise is radiated from the conductor exposed portion or noise is mixed in the conductor exposed portion from the outside.

Particularly, with improvement in an image quality of the endoscopic device, increase in pixels of the solid-state image pickup element has been promoted, and speed-up/capacity increase of the signal transmitted between the solid-state image pickup element and the processor has progressed. Thus, the problem of the high-frequency noise generated from the conductor exposed portion of the signal cable has become more remarkable.

### SUMMARY OF THE INVENTION

The present invention was made in view of the above circumstances and has an object to provide a solid-state image pickup device and an endoscopic device that can prevent radiation and mixing-in of noise in a conductor exposed portion at a distal end of a signal cable connected to a connection terminal of a circuit board while the size of an insertion portion of an endoscopic device is reduced.

In order to achieve the above object, a solid-state image pickup device according to the present invention includes a solid-state image pickup element, a circuit-board main body to which the solid-state image pickup element is connected, a shield piece consecutively connected to the circuit-board main body, capable of being folded, and having a shield pattern disposed, and a signal cable which is connected to a connection terminal disposed on the circuit-board main body and transmits an input/output signal to the solid-state image pickup element, and the shield piece is folded at least to one face side of a region where the connection terminal is formed.

According to the present invention, radiation or mixing-in of the noise generated in the conductor exposed portion of the signal cable connected to the connection terminal of the circuit-board main body is prevented by the shield pattern on the shield piece consecutively connected to the circuit-board main body. Also, shielding performance against the conductor exposed portion of the signal cable can be improved with a simple configuration, and a limited space at the distal end of the endoscope insertion portion can be effectively utilized without being subjected to the influence of the noise, and size reduction of the endoscope insertion portion can be promoted.

The solid-state image pickup device according to the present invention preferably includes the connection terminal in the shield pattern if the connection terminal is projected onto the same plane as the shield pattern disposed on the shield piece.

Also, the shield piece is preferably folded so as to become substantially in parallel with the circuit-board main body.

Also, it is preferable that at least two shield pieces are consecutively connected to the circuit-board main body, capable of being folded, and the two shield pieces are preferably folded so as to overlap each other.

Also, it is preferable that a shield layer is disposed on the signal cable side, an opening portion from which the shield pattern is exposed is formed in the shield piece, and the shield piece is folded so that the shield pattern exposed from the opening portion is in contact with the shield layer.

Also, it is preferable that a consecutive connecting portion that consecutively connects the circuit-board main body to the shield piece is disposed, and the shield pattern is extended to at least a part of the consecutive connecting portion.

Also, the circuit-board main body and the shield piece are preferably formed integrally from a flexible board having flexibility.

Also, peripheral portions of the shield piece and the connection terminal are preferably sealed and fixed by a resin.

Also, in order to achieve the above object, the endoscopic device according to the present invention is characterized by having the solid-state image pickup device described in any one of the first to eighth aspects at the distal end of the insertion portion to be inserted into the subject body.

The endoscopic device according to the present invention preferably includes a forceps channel communicating with a forceps outlet formed in the distal end of the insertion portion and through which a treatment instrument that performs a procedure for the subject body is inserted, and the shield piece is folded so as to be arranged between the connection terminal and the forceps channel.

According to the present invention, the radiation or mixing-in of the noise generated in the conductor exposed portion of the signal cable connected to the connection terminal of the circuit-board main body is prevented by the shield pattern of the shield piece consecutively connected to the circuit-board main body. Also, the shielding performance against the conductor exposed portion of the signal cable can be improved with a simple configuration, and a limited space at the distal end of the endoscope insertion portion can be effectively utilized without being subjected to the influence of the noise, and size reduction of the endoscope insertion portion can be promoted.

### BRIEF DESCRIPTION OF THE DRAWINGS

FIG. 1 is an entire configuration diagram illustrating an electronic endoscopic system;

FIG. 2 is a sectional view illustrating the inside of a flexible portion;

FIG. 3 is a plan view illustrating a distal end face of a distal end portion;

FIG. 4 is an outline sectional view of the inside of the distal end position when seen from the side face;

FIG. 5 is a configuration diagram illustrating another arrangement example of a solid-state image pickup element;

FIG. 6 is a sectional view illustrating the inside of a multi-core cable;

FIG. 7 is a perspective view illustrating a configuration example of a flexible board and a peripheral portion thereof;

FIG. 8 is an extended plan view illustrating a state before the flexible board is folded;

FIG. 9 is an extended plan view illustrating another configuration example of the flexible board;

FIG. 10 is a sectional view of the flexible board shown in FIG. 9 when being folded, seen from the front;

FIG. 11 is an extended plan view illustrating still another configuration example of the flexible board;

FIG. 12 is a sectional view of the flexible board shown in FIG. 10 when being folded, seen from the front; and

FIG. 13 is a side view illustrating a configuration of a flexible board and a peripheral portion thereof according to a second embodiment.

### DETAILED DESCRIPTION OF THE PREFERRED EMBODIMENTS

Preferred embodiments of a solid-state image pickup device and an endoscopic device according to the present invention will be described below referring to the attached drawings.

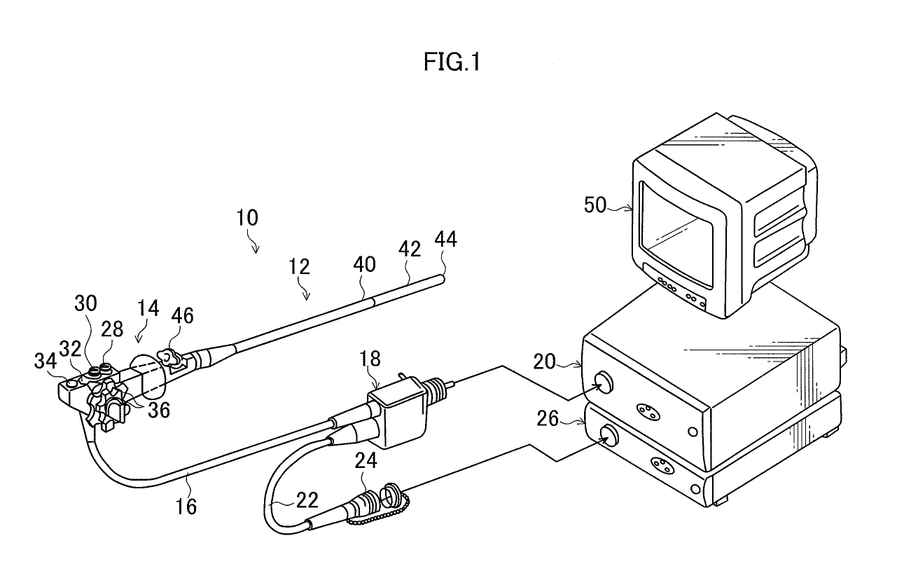

FIG. 1 is an entire configuration diagram illustrating an endoscopic system. The endoscopic system shown in FIG. 1 mainly includes an endoscopic device (electronic endoscope) 10 on which a solid-state image pickup device to which the present invention is applied is mounted, a processor 26, a light source device 20, and a monitor device 50.

The endoscopic device 10 mainly includes an insertion portion 12 to be inserted into a body cavity of a patient (subject) and a hand operation portion 14 consecutively connected to a base end portion of the insertion portion 12.

In the hand operation portion 14, an air/water feed button 28, a suction button 30, a shutter button 32, a function switching button 34, and a pair of angle knobs 36 and 36 are disposed. Also, a forceps inlet 46 through which a treatment instrument such as forceps are inserted is disposed.

Also, in the hand operation portion 14, an LG connector 18 is disposed through a universal cable 16, and the LG connector 18 is detachably joined to a light source device 20. Also, to the LG connector 18, an electric connector 24 is connected through a cable 22, and the electric connector 24 is detachably joined to a processor 26.

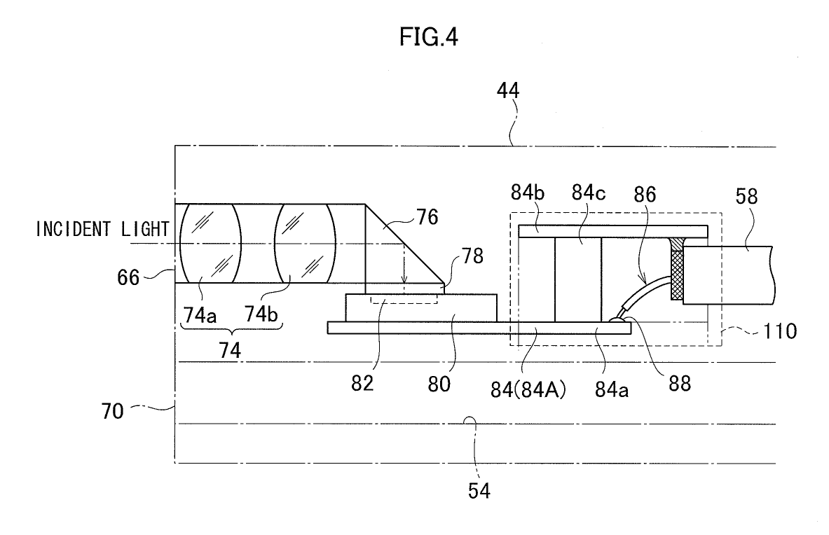

The insertion portion 12 is composed of a distal end portion 44, a bent portion 42, and a flexible portion 40 in the order from the distal end (opposite to the hand operation portion 14). The distal end portion 44 is formed by a hard metal material or the like and incorporates a solid-state image pickup element (shown in FIG. 4 by reference numeral 80) for imaging the inside of the subject.

The bent portion 42 is composed by connecting a plurality of bent pieces and bent and operated vertically and horizontally by pushing/pulling a wire inserted and set in the insertion portion 12 in conjunction with the operation of the angle knobs 36 and 36 disposed on the hand operation portion 14. As a result, the distal end portion 44 is directed to a desired direction in the subject.

The flexible portion 40 is a lengthy portion with a small diameter that connects the hand operation portion 14 and the bent portion 42 to each other and has flexibility. The flexible portion 40 has a length of 1 meter to several meters so that the distal end portion 44 can reach the portion to be observed and that a distance from a patient is kept to such a degree that grasping and operation of the hand operation portion 14 by an operator is not interfered.

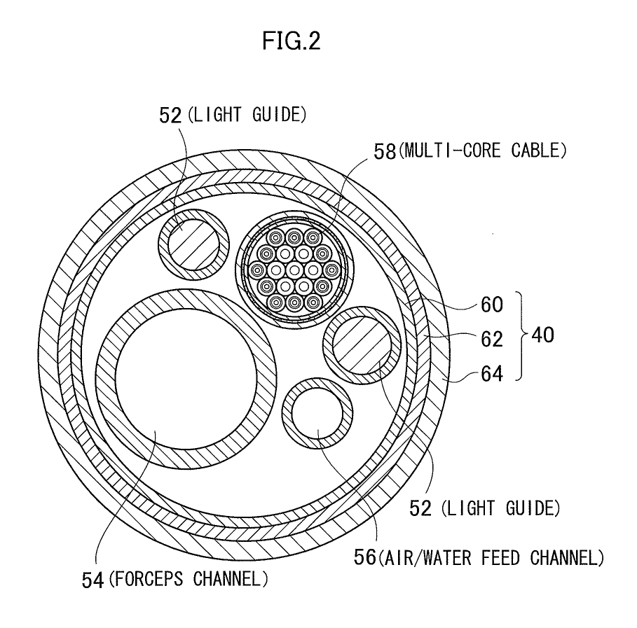

FIG. 2 is a sectional view illustrating the inside of the flexible portion 40. As shown in FIG. 2, inside the flexible portion 40 is constituted such that a plurality of contents, that is, light guides 52 and 52 that lead illumination light, a forceps channel 54, an air/water feed channel 56, a multi-core cable 58 and the like are freely inserted therethrough.

The flexible portion 40 is composed of three layers, that is, a spiral tube 60 called a flex that protects the inside while keeping flexibility, a net 62 called a braid that covers the spiral tube 60 and prevents elongation of the spiral tube 60, and an outer layer 64 that wraps the net 62 with a resin in the order from the inside.

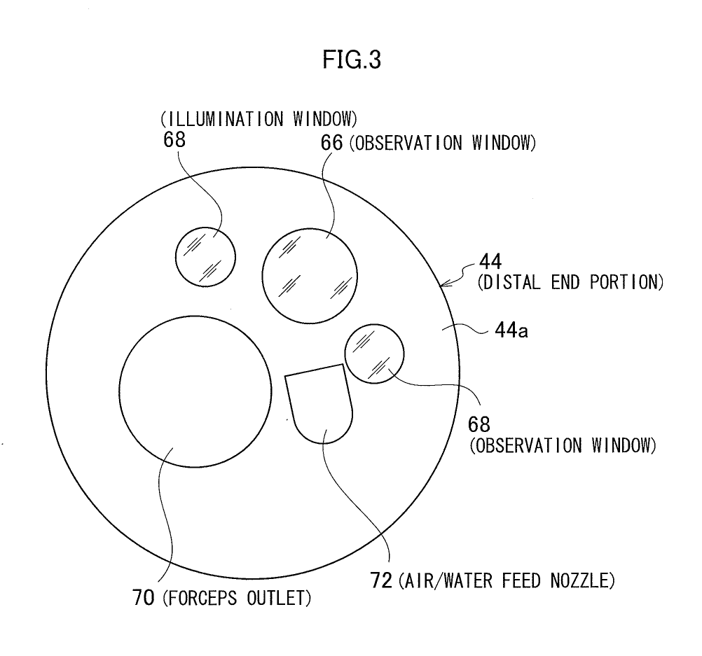

Subsequently, the structure of the distal end portion 44 will be described. FIG. 3 is a plan view illustrating a distal end face of the distal end portion 44, and FIG. 4 is an outline sectional view of the inside of the distal end portion 44 when seen from the side face.

As shown in FIG. 3, on a distal end face 44a of the distal end portion 44, an observation window 66, illumination windows 68 and 68, a forceps outlet 70, an air/water feed nozzle 72 and the like are disposed in an exposed manner.

Behind the illumination windows 68 and 68, a lens for illumination is incorporated so as to lead illumination light emitted from the light source device 20 by the light guides 52 and 52 and to radiate the light to the portion to be observed in the body cavity. The forceps outlet 70 is made to communicate with the forceps inlet 46 disposed in the hand operation portion 14 through the forceps channel 54. The air/water feed nozzle 72 injects washing water or air for washing off stains on the observation window 66 by operating the air/water feed button 28 disposed on the hand operation portion 14.

In the rear of the observation window 66, as shown in FIG. 4, an objective optical system 74 that collects image light (incident light) of the portion to be observed taken in through the observation window 66 is disposed. The objective optical system 74 is composed of a plurality of lenses 74a and 74b held in a lens barrel, not shown. In the rear of the objective optical system 74, a prism 76 that converts the direction of an optical path of the incident light having passed through the objective optical system 74 by 90 degrees is disposed, and below that, a solid-state image pickup element 80 provided with a cover glass 78 is disposed. The cover glass 78 is a transparent protective member that protects an image pickup face (light receiving portion) 82 of the solid-state image pickup element 80 and is arranged on the image pickup face 82 through a spacer, not shown.

The image light of the portion to be observed taken in through the observation window 66 is formed on the image pickup face 82 of the solid-state image pickup element 80 through the objective optical system 74, the prism 76, and the cover glass 78.

The solid-state image pickup element 80 is composed of an interline type CCD, for example, made in a form of a non-packaged bare chip and connected to an electrode on a flexible printed circuit board (FPC) 84, in which the electrode on the chip has flexibility, by methods including wire bonding, TAB (tape automated bonding), flip chip and the like.

As shown in FIG. 4, on the rear end side of the flexible board 84, an input/output terminal (connection terminal) 88 to which a plurality of signal lines 86 constituting the multi-core cable 58 are soldered is disposed. In FIG. 4, in order to avoid complexity in the figure, only one signal line 86 is shown.

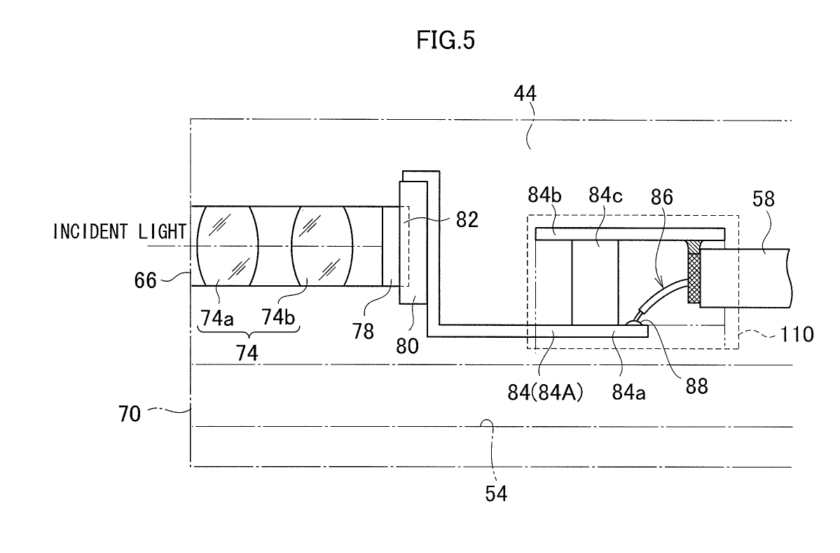

In FIG. 4, the image pickup face 82 of the solid-state image pickup element 80 is arranged so as to be in parallel with the axial direction of the distal end portion 44, but not limited to that, the face may be arranged perpendicularly to the axial direction of the distal end portion 44 as shown in FIG. 5.

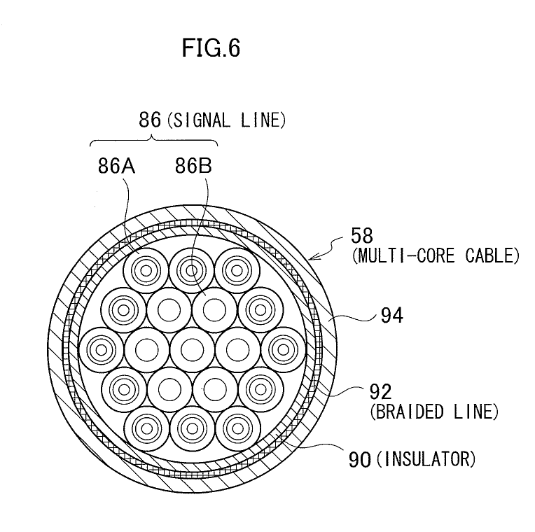

FIG. 6 is a sectional view illustrating the inside of the multi-core cable. As shown in FIG. 6, the multi-core cable 58 is constituted by bundling a plurality of the signal lines 86, wrapping the bundled signal lines 86 with an insulator 90, wrapping the insulator 90 with a braided line 92 as an electric shield layer, and by wrapping the braided line 92 with an outer sheath 94. The signal lines 86 include a coaxial line 86A and an insulating line 86B, and if a frequency in use is a high frequency, the coaxial line 86A is used, while in the case of a low frequency, the insulating line 86B is used. The multi-core cable 58 has the insulator 90, the braided line 92, and the outer sheath 94 peeled off in the vicinity of the flexible board 84 so as to expose the plurality of signal lines 86. To the input/output terminal 88 of the flexible board 84, the conductor whose insulator as the outer sheath of the signal line 86 is peeled off is connected.

Here, a configuration of the flexible board 84 will be described in detail.

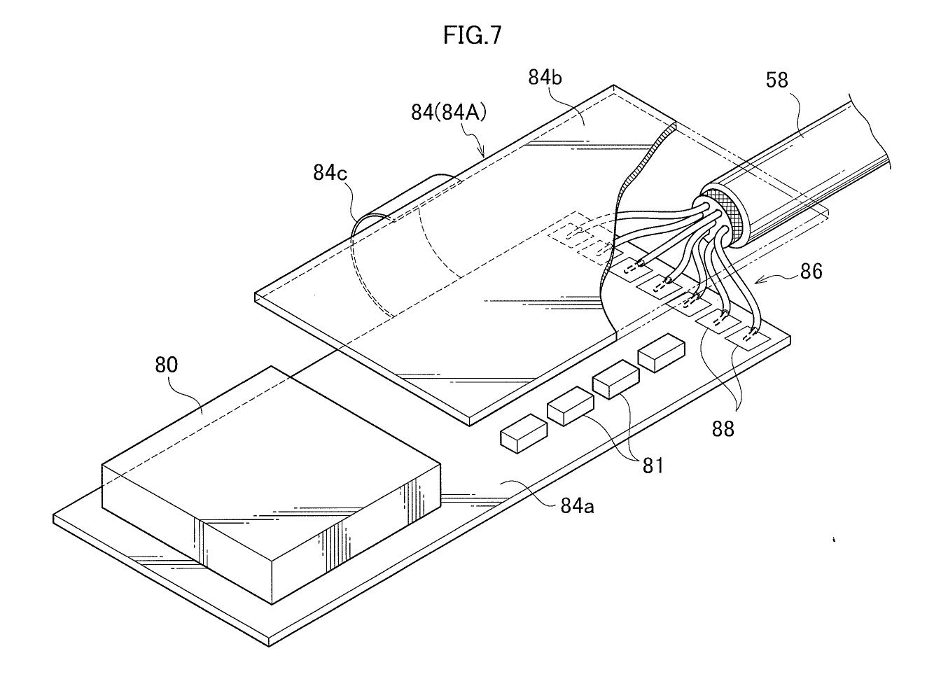

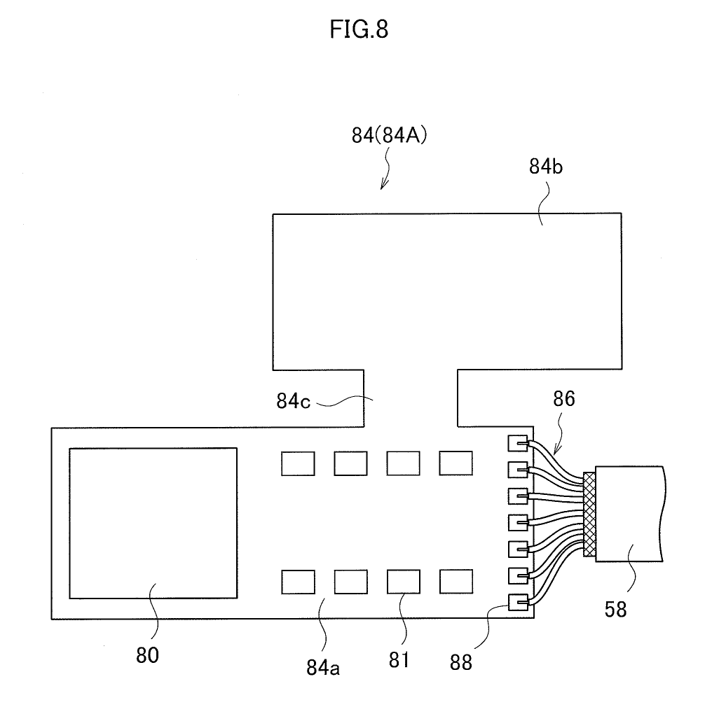

FIG. 7 is a perspective view illustrating configurations of the flexible board and a peripheral portion thereof, and FIG. 8 is an extended plan view illustrating a state before the flexible board shown in FIG. 7 is folded.

As shown in FIGS. 7 and 8, the flexible board 84 (84A) of the first embodiment is integrally composed of a board main body portion (circuit-board main body) 84a on which the solid-state image pickup element 80 is mounted, a small piece portion (shield piece) 84b having the width equal to that of the board main body portion 84a and formed protruding rearward from the rear end of the board main body portion 84a, and a consecutive connection portion 84c that connects the board main body portion 84a and the small piece portion 84b to each other. On the flexible board 84, a plurality of electronic components 81 (such as an IC, a resistor, a capacitor, a transistor and the like) are disposed.

The both front and back surfaces of the flexible board 84 (excluding the terminal portion) are composed of an insulating member having flexibility (an insulating film such as a polyimide film, for example), and first and second conductor patterns are embedded inside thereof.

The first conductor pattern is disposed inside the board main body portion 84a, whose one end is electrically connected to the solid-state image pickup element, while the other end is electrically connected to the input/output terminal 88, and functions as a wiring pattern that transmits an electric signal inputted/outputted with respect to the solid-state image pickup element 80 to the input/output terminal 88 and the like.

The second conductor pattern is disposed inside the small piece portion 84b and functions as a shield pattern that prevents radiation and mixing-in of noise in the conductor exposed portion at the distal end of the signal line 86 connected to the input/output terminal 88 of the board main body portion 84a. The shield pattern may be formed flat on the entire surface of the small piece portion 84b or may be formed in the mesh state. One end of the shield pattern is electrically connected to a shield terminal (not shown) disposed on the small piece portion 84b. The shield pattern is not limited to the small piece portion 84b but also may be extended to a part or the whole of the consecutive connection portion 84c.

The flexible board 84 configured as above is folded in the U shape (or a squared U) so that the board main body portion 84a and the small piece portion 84b become substantially parallel and a plurality of input/output terminals 88 (that is, the distal end portions of the plurality of signal lines 86 exposed from the multi-core cable 58) are arranged in a region surrounded by the board main body portion 84a and the small piece portion 84b.

In other words, in a state in which the flexible board 84 is folded as shown in FIG. 7, if each input/output terminal 88 is projected onto the same plane as the small piece portion 84b, the input/output terminals 88 are contained in the region in which the shield pattern disposed on the small piece portion 84b is formed.

Also, in the state in which the flexible board 84 is folded, at least a region 110 (a region surrounded by a dotted line in FIG. 4) including the small piece portion 84b and the input/output terminals 88 is preferably sealed and fixed by a resin. Then, the positional relationship between the small piece portion 84b and the input/output terminal 88 can be reliably fixed.

According to this embodiment, since above the distal end portions (conductor exposed portions) of the plurality of signal lines 86 exposed from the multi-core cable 58, the small piece portion 84b formed integrally with the board main body portion 84a constituting the flexible board 84 is arranged, radiation and mixing-in of the noise in the conductor exposed portion of the signal lines 86 can be reliably prevented by the shield pattern formed on the small piece portion 84b. As a result, without being subjected to the noise influence, a space on the side opposite (upper side in FIG. 4) to the input/output terminal 88 side with the small piece portion 84b between them can be effectively utilized, and the size of the distal end portion 44 can be reduced.

The flexible board 84 of this embodiment is not limited to the configuration shown in FIGS. 7 and 8. Another configuration example of the flexible board 84 will be described below.

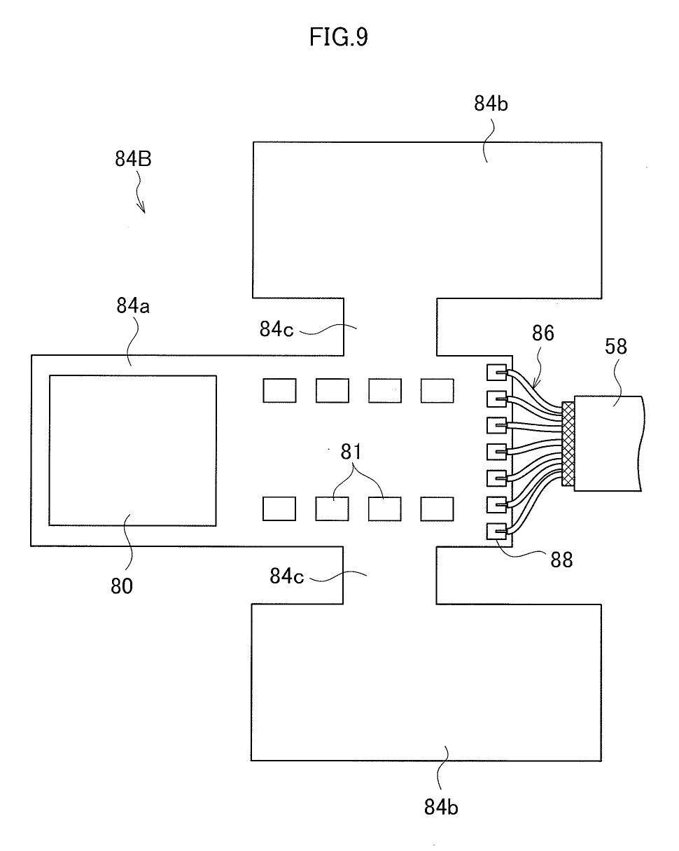

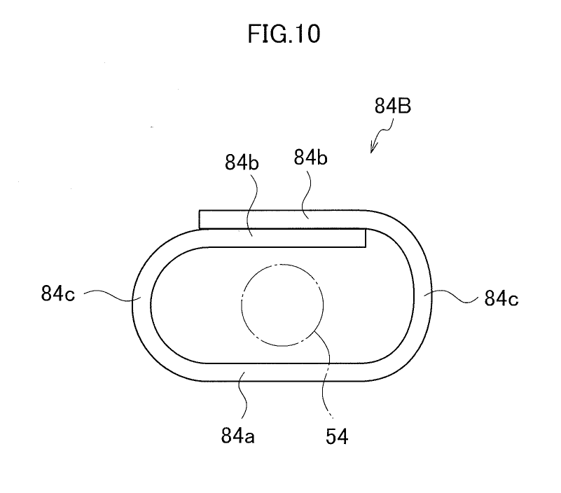

FIG. 9 is an extended plan view illustrating another configuration example of the flexible board, and FIG. 10 is a sectional view of the flexible board shown in FIG. 9, which is folded and seen from the front.

A flexible board 84B shown in FIG. 9 has the small piece portion 84b connected not only to one side but the both sides of the board main body portion 84a through consecutive connection portions 84c, respectively. Thus, if the flexible board 84 is folded as shown in FIG. 10, the shield patterns formed on the two small piece portions 84b and 84b overlap each other vertically, shielding performance against the distal end portion (conductor exposed portion) of the signal line 86 is improved, and radiation and mixing-in of the noise can be prevented more reliably.

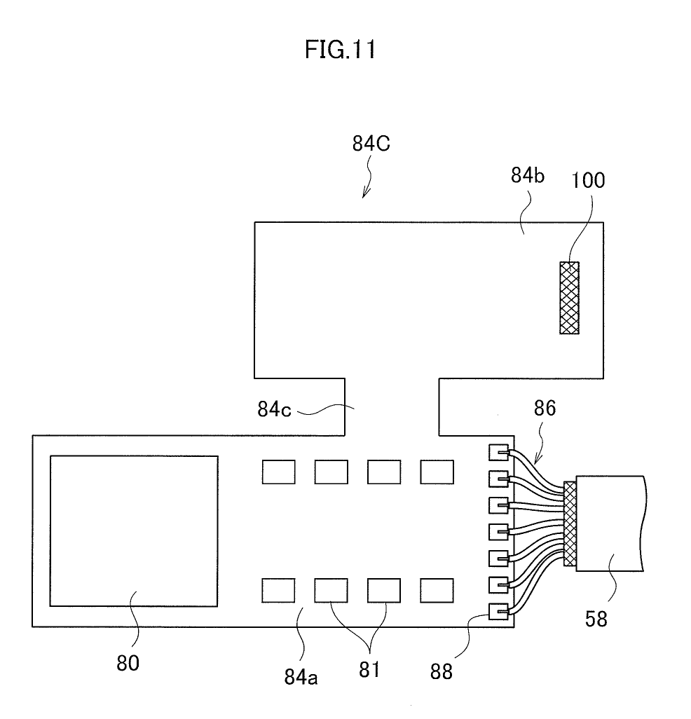

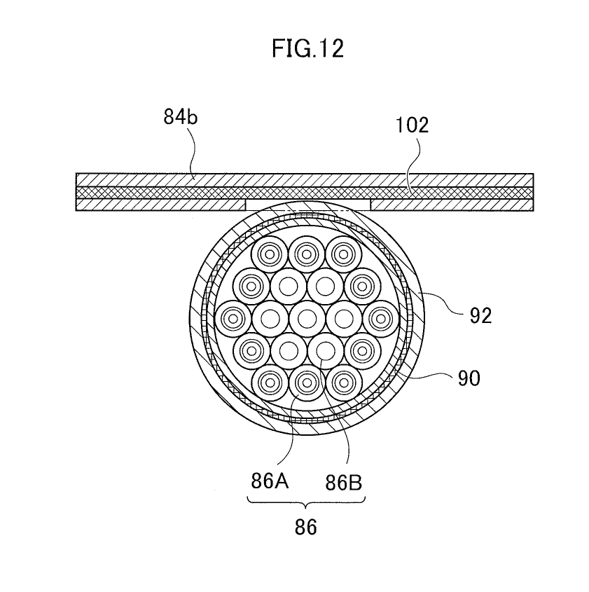

FIG. 11 is an extended plan view illustrating still another configuration example of the flexible board, and FIG. 12 is a sectional view of the flexible board shown in FIG. 11, which is folded and seen from the front.

In a flexible board 84C shown in FIG. 11, an opening portion 100 from which a shield pattern is exposed to the surface is formed at a predetermined position on the rear end side of the small piece portion 84b. This opening portion 100 functions as a shield terminal and is configured so as to be electrically conducted by bringing a shield pattern 102 exposed from the opening portion 100 into direct contact with a braided line (electric shield layer) 92 exposed from the distal end of the multi-core cable 58 when the flexible board 84 is folded as shown in FIG. 12.

Also, if the coaxial line 86A is used as the signal line 86, electric conductivity may be accomplished by bringing a braided line (shield layer) disposed in the coaxial line 86A into direct contact with the shield pattern 102 exposed from the opening portion 100, instead of the braided line 92 of the multi-core cable 58.

According to this configuration, since wiring that connects the shield pattern of the small piece portion 84b is no longer needed, the internal structure of the distal end portion 44 can be simplified, and the size of the distal end portion 44 can be reduced.

### Second Embodiment

Subsequently, a second embodiment of the present invention will be described. The description will be omitted below for the portions in common with the first embodiment, and the characteristic portions of this embodiment will be mainly described.

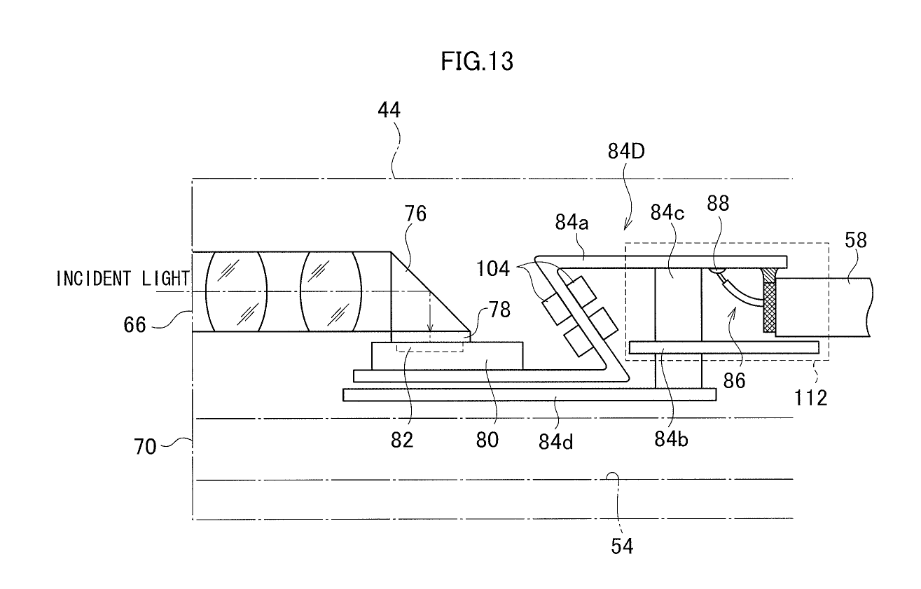

FIG. 13 is a side view illustrating configurations of a flexible board and a peripheral portion thereof according to the second embodiment. In FIG. 13, members in common with or similar to those in FIG. 4 are given the same reference numerals, and the description will be omitted.

As shown in FIG. 13, a flexible board 84D of the second embodiment is similar to the first embodiment in the point that it is composed of the board main body portion 84a, the small piece portion 84b, and the consecutive connection portion 84c, but the board main body portion 84a is folded in the Z shape, and on the front and back faces of the folded portion, a plurality of electronic components 104 (such as an IC, a capacitor, a resistor, a transistor and the like) are mounted with high density.

Also, the input/output terminal 88 formed on the rear end side of the board main body portion 84a is formed on the side (back face side) opposite to the face on which the solid-state image pickup element 80 is mounted. Thus, the plurality of signal lines 86 constituting the multi-core cable 58 are connected to the back face side of the board main body portion 84a, and the small piece portion 84b connected to the board main body portion 84a through the consecutive connection portion 84c is folded downward from the board main body portion 84a on the side opposite to that in the first embodiment.

Also, in the state in which the flexible board 84D is folded, a region 112 containing at least the small piece portion 84b and the input/output terminal 88 (a region surrounded by a dotted line in FIG. 13) is preferably sealed and fixed by a seal.

Also, as shown in FIG. 13, the flexible board 84D may be further provided with a second small piece portion (second shield piece) 84d so that the second small piece portion 84d is arranged between the face opposite to the face on which the solid-state image pickup element 80 of the flexible board 84D is arranged and the forceps channel 54.

According to this embodiment, the effect similar to that in the first embodiment can be obtained, and since the small piece portion 84b integrally formed with the board main body portion 84a constituting the flexible board 84 is arranged between the input/output terminal 88 of the board main body portion 84a and the forceps channel 54, influence by the high-frequency noise radiated from an electric treatment instrument such as an electric knife inserted into the forceps channel 54 can be also prevented.

The solid-state image pickup device and the endoscopic device of the present invention have been described above in detail, but the present invention is not limited to the above examples, and it is needless to say that various improvements and deformations can be made within a range not departing from the gist of the present invention.

What is claimed is:

1. A solid-state image pickup device, comprising:
a solid-state image pickup element;
a circuit-board main body to which the solid-state image pickup element is connected;
a shield piece consecutively connected to the circuit-board main body, capable of being folded, and having a shield pattern disposed; and
a signal cable which is connected to a connection terminal disposed on the circuit-board main body and transmits an input/output signal to the solid-state image pickup element, wherein
the shield piece is folded at least to one face side of a region where the connection terminal is formed.

2. The solid-state image pickup device according to claim 1, wherein
if the connection terminal is projected onto the same plane as the shield pattern disposed on the shield piece, the connection terminal is contained within the shield pattern.

3. The solid-state image pickup device according to claim 1, wherein
the shield piece is folded so as to become substantially in parallel with the circuit-board main body.

4. The solid-state image pickup device according to claim 1, wherein
at least two shield pieces are consecutively connected to the circuit-board main body, capable of being folded; and
the two shield pieces are folded so as to overlap each other.

5. The solid-state image pickup device according to claim 1, wherein
a shield layer is disposed on the signal cable side;
an opening portion from which the shield pattern is exposed is formed in the shield piece; and
the shield piece is folded so that the shield pattern exposed from the opening portion is in contact with the shield layer.

6. The solid-state image pickup device according to claim 1, further comprising:
a consecutive connecting portion that consecutively connects the circuit-board main body to the shield piece, where
the shield pattern is extended to at least a part of the consecutive connecting portion.

7. The solid-state image pickup device according to claim 1, wherein
the circuit-board main body and the shield piece are formed integrally from a flexible board having flexibility.

8. The solid-state image pickup device according to claim 1, wherein
peripheral portions of the shield piece and the connection terminal are sealed and fixed by a resin.

9. An endoscopic device including the solid-state image pickup device according to claim 1 at a distal end of an insertion portion to be inserted into a subject body.

10. The endoscopic device according to claim 9, further comprising:
a forceps channel communicating with a forceps outlet formed in the distal end of the insertion portion and through which a treatment instrument that performs a procedure on the subject body is inserted, wherein
the shield piece is folded so as to be arranged between the connection terminal and the forceps channel.
